(19) United States
(12) Patent Application Publication
(10) Pub. No.: US 2007/0154438 A1
(43) Pub. Date: Jul. 5, 2007

(54) Method for producing a water-in-water polyvinyl lactam dispersion with a k value of = 120

(75) Inventors: Lysander Chrisstoffels, Limburgerhof (DE); Ralf Widmaier, Mannheim (DE); Ivette Garcia Castro, Ludwigshafen Gartenstadt (DE); Ludger Wegmann, Ludwigshafen (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 10/591,654

(22) PCT Filed: Apr. 14, 2005
(86) PCT No.: PCT/EP05/03915

§ 371 Date: Sep. 5, 2006

(30) Foreign Application Priority Data
Apr. 16, 2004 (DE) 10 2004 019 179.4

(51) Int. Cl. A61K 8/81; C08F 26/10; C08F 26/08

(52) U.S. Cl. 424/70.15; 526/264; 524/516

(57) ABSTRACT

Method for producing water-in-water polyvinyl lactam dispersions with a K value of =120 by radical polymerisation.

The present invention provides a process for preparing a water-in-water dispersion of polyvinyllactam having a K value of ≧120 by free-radically initiated polymerization of at least one N-vinyllactam of general formula I 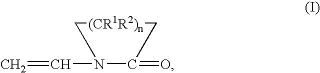
where
R¹, R² independently of one another are hydrogen and/or C₁-C₈ alkyl, and
n is an integer from 2 to 8,
in an aqueous reaction medium,
wherein said at least one N-vinyllactam I used for the polymerization is composed of at least 50% by weight of N-vinyl-2-pyrrolidone (R¹ and R² as hydrogen, n as 3), the polymerization temperature is ≦70° C. and the free-radically initiated polymerization of said at least one N-vinyllactam I takes place in the presence of
a) from 1% to 100% by weight, based on the saturation amount in the aqueous reaction medium, of at least one organic or inorganic salt,
b) from 0.1% to 30% by weight of at least one polymeric anionic dispersant, based on the total amount of said at least one N-vinyllactam I used for the polymerization, and
c) from 0.01% to 0.25% by weight of at least one free-radical initiator, based on the total amount of said at least one N-vinyllactam I used for the polymerization,
   and the reaction conditions are chosen so that during the polymerization reaction at least a portion of said at least one N-vinyllactam I and of the polyvinyllactam formed therefrom by polymerization are present in the form of a separate phase in the aqueous reaction medium.

The following prior art is a starting point for preparing high molecular mass polyvinyllactam compounds, especially poly-N-vinyl-2-pyrrolidone (PVP).

Thus WO 91/15522 discloses the preparation of water-soluble PVP with K values >120 by free-radical aqueous solution polymerization of N-vinyl-2-pyrrolidone (VP). Characteristic of the process is that in the course of VP polymerization water is added so that the viscosity of the resulting PVP solution does not increase too greatly.

WO 91/03496 discloses the solution polymerization by means of tert-amyl peroxypivalate as free-radical initiator. Besides copolymers of maleic anhydride and alkyl vinyl ethers the preparation of PVP having a K value of approximately 120 is also described. The PVP content of the high-viscosity aqueous solution disclosed by way of example is approximately 21% by weight.

WO 94/18241 discloses the preparation of PVP having K values in the range from 30 to 150 in the form of high-viscosity aqueous solutions by polymerizing VP using a specific free-radical initiator system composed of at least two free-radical initiators whose 1-hour half-life temperatures differ by more than 5° C. Only one example, however, is given of the preparation of PVP having a K value >100. The PVP content of this aqueous PVP solution, however, is only about 19% by weight.

WO 94/22953 relates to the preparation of PVP having a K value of from 15 to 130 in the form of aqueous solutions by free-radically polymerizing VP or oligomers thereof using as free-radical initiator 2,2′-azobis(2-methylbutanenitrile). The aqueous PVP solutions obtained, with K values of >120, have PVP contents of only about 20% by weight, however.

A disadvantage of these processes is that the preparation of these high molecular mass PVP polymers by solution polymerization leads to high viscosities of the PVP solutions even at relatively low PVP contents. The poorer space/time yields which this entails result in high production costs. Considered a further disadvantage of the prior art processes are the relatively high fractions of undissolved gel particles, which lead to a multiplicity of disadvantages both in the production operation (longer filtering and dispensing times) and on subsequent application of the PVP polymers (inhomogeneities in the corresponding formulations).

It was an object of the present invention to provide an improved polymerization process for high molecular mass N-vinyllactams having a K value >120, especially N-vinyl-2-pyrrolidone, which exhibits improved space/time yields and opens up a route to low-viscosity aqueous systems combining higher polyvinyllactam contents with lower gel contents.

The process defined at the outset has been found accordingly. Processes for preparing water-in-water polymer dispersions by free-radically induced polymerization of ethylenically unsaturated compounds (monomers) are general knowledge (see for example WO 98/31748, WO 98/54234, EP-A 630909, EP-A 984990 or U.S. Pat. No. 4,380,600).

In accordance with the invention at least one N-vinyllactam of general formula I 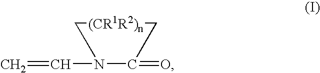
where
R¹, R² independently of one another are hydrogen and/or C₁-C₈ alkyl, and
n is an integer from 2 to 8,
are used for the polymerization in an aqueous reaction medium, said at least one N-vinyllactam I being composed of at least 50% by weight of N-vinyl-2-pyrrolidone (R¹ and R² as hydrogen, n as 3).

R¹ and R² in this formula can independently of one another be hydrogen and/or C₁-C₈ alkyl, for example methyl, ethyl, n-propyl, isopropyl, n-butyl, isobutyl, tert-butyl and also n-pentyl, n-hexyl, n-heptyl or n-octyl and their isomeric alkyl groups. R¹ and R² are preferably hydrogen and methyl. Particular preference is given to hydrogen. In many cases the N-vinyllactam I contains no methyl groups or only one in all.

In accordance with the invention n is an integer from 2 to 8, frequently 3, 4, 5, 6 and 7. In particular n is 3 and 5.

Examples of N-vinyllactams I which can be used with advantage in accordance with the invention are the N-vinyl derivatives of the following lactams: 2-pyrrolidone, 2-piperidone, ε-caprolactam and their alkyl derivatives, such as 3-methyl-2-pyrrolidone, 4-methyl-2-pyrrolidone, 5-methyl-2-pyrrolidone, 3-ethyl-2-pyrrolidone, 3-propyl-2-pyrrolidone, 3-butyl-2-pyrrolidone, 3,3-dimethyl-2-pyrrolidone, 3,5-dimethyl-2-pyrrolidone, 5,5-dimethyl-2-pyrrolidone, 3,3,5-trimethyl-2-pyrrolidone, 5-methyl-5-ethyl-2-pyrrolidone, 3,4,5-trimethyl-2-pyrrolidone, 3-methyl-2-piperidone, 4-methyl-2-piperidone, 5-methyl-2-piperidone, 6-methyl-2-piperidone, 6-ethyl-2-piperidone, 3,5-dimethyl-2-piperidone, 4,4-dimethyl-2-piperidone, 3-methyl-ε-caprolactam, 4-methyl-ε-caprolactam, 5-methyl-ε-caprolactam, 6-methyl-ε-caprolactam, 7-methyl-ε-caprolactam, 3-ethyl-ε-caprolactam, 3-propyl-ε-caprolactam, 3-butyl-ε-caprolactam, 3,3-dimethyl-ε-caprolactam or 7,7-dimethyl-ε-caprolactam. It will be appreciated that mixtures of aforementioned N-vinyllactams I can also be used.

Said at least one N-vinyllactam I for polymerization is composed of at least 50% by weight of VP. Often said at least one N-vinyllactam I is composed of ≧60%, ≧70%, ≧80%, ≧90% or even 100% by weight and all values in between, of VP. Frequently VP is used exclusively for the polymerization.

In accordance with the invention the entirety of N-vinyllactam I can be introduced as an initial charge in the reaction medium. It is also possible, however, to introduce only a portion of said at least one N-vinyllactam I as an initial charge in the reaction medium and to supply the remainder if appropriate or the entirety of the N-vinyllactam I to the reaction medium under polymerization conditions.

The process of the invention is conducted in the presence of from 1% to 100% by weight, based on the saturation amount in the aqueous reaction medium, of at least one organic or inorganic salt. The function of said at least one salt is to lower the solubility both of N-vinyllactam I and of the polyvinyllactam formed, so that at least a portion of the N-vinyllactam I used for the polymerization and of the polyvinyllactam formed therefrom by polymerization is present as a separate heterogeneous phase in the aqueous reaction medium under polymerization conditions.

The selection of the salt in question depends essentially on the N-vinyllactam I employed, the polyvinyllactam to be produced, and the polymeric anionic dispersant employed, and also if appropriate further auxiliaries. The selection of the identity and quantity of the salt is made such that under polymerization conditions (temperature, pressure, presence of auxiliaries if appropriate, etc.) not only at least a portion of the N-vinyllactam I used for the polymerization but also at least one portion of the polyvinyllactam formed are present as a separate heterogeneous phase in the salt solution. It is advantageous in accordance with the invention the higher the fraction of N-vinyllactam I and polyvinyllactam present as a separate phase. It is advantageous if under polymerization conditions ≧60%, frequently ≧70% and often ≧80% by weight of the at least one unreacted N-vinyllactam I and also ≧70%, frequently ≧80% and often ≧90% by weight of the polyvinyllactam formed are present as a separate heterogeneous phase in the aqueous reaction medium.

The salts to be employed that can be used for the process of the invention are described exhaustively in WO 98/14405 and WO 00/20470, which are hereby incorporated by reference.

Suitable salts are inorganic salts, preferably cosmotropic salts, such as fluorides, chlorides, sulfates, phosphates or hydrogenphosphates of metal ions or ammonium ions. Typical representatives are sodium sulfate, potassium sulfate, ammonium sulfate, magnesium sulfate, aluminum sulfate, sodium chloride, potassium chloride, sodium dihydrogenphosphate, diammonium hydrogenphosphate, dipotassium hydrogenphosphate, calcium phosphate, sodium citrate and iron sulfate.

Chaotropic salts, such as thiocyanates, perchlorates, chlorates, nitrates, bromides and iodides, can likewise be used. Typical representatives are calcium nitrate, sodium nitrate, ammonium nitrate, aluminum nitrate, sodium thiocyanate and sodium iodide.

It is advantageous to use salts of organic C₁ to C₁₅ carboxylic acids, especially the alkali metal salts, sodium or potassium salts for example, or ammonium salts of monobasic, dibasic or polybasic organic C₁ to C₁₂ carboxylic acids, such as formic acid, acetic acid, citric acid, oxalic acid, malonic acid, succinic acid, adipic acid, suberic acid, phthalic acid, agaricic acid, trimesic acid, 1,2,3-propanetricarboxylic acid and also 1,4-, 2,3- or 2,6-naphthalenedicarboxylic acid, for example.

The aforementioned salts can be used individually or as mixtures of two or more salts. Often a mixture of two or more salts is more effective than one salt alone, based on the amount employed.

The salts are added in an amount which is from 1% to 100%, preferably from 10% to 90% and more preferably from 15% to 75% by weight of the saturation amount in the aqueous reaction medium under reaction conditions.

By 100% by weight saturation amount in the reaction medium is meant the amount of salt or salts which still just dissolves, without precipitating, in the aqueous reaction medium of the employed N-vinyllactam I in the presence of said at least one polymeric anionic dispersant and also if appropriate of further auxiliaries, and at the reaction temperature employed.

In accordance with the invention it is possible for the entirety of said at least one salt to be included in the initial charge in the reaction medium. An alternative possibility is to introduce if appropriate only a portion of said at least one salt as an initial charge in the reaction medium and to supply the remainder if appropriate or the entirety of said at least one salt to the reaction medium under polymerization conditions. In that case, however, it is necessary to ensure that not only the N-vinyllactam I used for the polymerization (up until the time of its reaction) but also the polyvinyllactam formed are always in the form of a separate heterogeneous phase in the aqueous reaction medium under reaction conditions.

The process of the invention takes place in the presence of from 0.1% to 30%, often from 0.5% to 20% and frequently from 1% to 10% by weight of at least one polymeric anionic dispersant, based in each case on the total amount of said at least one N-vinyllactam I used for the polymerization.

A polymeric anionic dispersant for the purposes of this text embraces all polymeric compounds whose average molecular weight is >1000 g/mol and whose actively dispersing polymer framework carries anionic groups. Frequently the average molecular weight is from 1500 to 3 000 000 g/mol or from 10 000 to 2 000 000 g/mol and often from 30 000 to 1 500 000, determined in each case by means of standard methods of gel permeation chromatography.

Suitable polymeric anionic dispersants include in particular homopolymers and copolymers of the following monomers: acrylic acid, methacrylic acid, crotonic acid, ethylacrylic acid, itaconic acid, 2-acrylamido-2-methylpropanesulfonic acid, vinylsulfonic acid, vinylsulfuric acid, vinylphosphoric acid, 10-undecenoic acid, 4-pentenoic acid, cinnamic acid, maleic acid, maleic anhydride, fumaric acid, 3-butenoic acid, 5-hexenoic acid, 6-heptenoic acid, 7-octenoic acid, citraconic acid, mesaconic acid, styrenesulfonic acid, styrenesulfuric acid, 3-sulfopropyl acrylate, bis-(3-sulfopropyl) itaconate, 3-sulfopropyl methacrylate, 3-allyloxy-2-hydroxypropane-1-sulfonic acid, 2-acrylamido-2-methylethanesulfonic acid, 2-sulfoethyl acrylate, bis-(2-sulfoethyl) itaconate, 2-sulfoethyl methacrylate, 3-sulfopropyl methacrylate, 3-allyloxy-2-hydroxypropane-1-sulfonic acid, 3-allyloxy-2-hydroxyethane-1-sulfonic acid and also their alkaline metal and ammonium salts, in particular their sodium and potassium salts.

Besides the aforementioned acid-functional monomers the polymeric anionic dispersants in the form of their copolymers may also include the following neutral monomers in copolymerized form: ethylene, isobutene, vinylaromatic monomers, such as styrene, α-methylstyrene, o-chlorostyrene or vinyltoluenes, vinyl halides, such as vinyl chloride or vinylidene chloride, ethers of vinyl alcohol and monoalcohols containing 1 to 18 carbon atoms, such as methyl vinyl ether, esters of vinyl alcohol and monocarboxylic acids containing 1 to 18 carbon atoms, such as vinyl acetate, vinyl propionate, vinyl n-butyrate, vinyl laurate and vinyl stearate, esters of preferably C3 to C6 α,β-monoethylenically unsaturated monocarboxylic and dicarboxylic acids, such as especially acrylic acid, methacrylic acid, maleic acid, fumaric acid and itaconic acid, with generally C1 to C12, preferable C1 to C8 and especially C1 to C4 alkanols, such as particularly methyl, ethyl, n-butyl, isobutyl, pentyl, hexyl, heptyl, octyl, nonyl, decyl and 2-ethylhexyl acrylate and methacrylate, dimethyl or di-n-butyl fumarate and maleate, nitriles of α,β-monoethylenically unsaturated carboxylic acids, such as acrylonitrile, methacrylonitrile, fumaraonitrile, maleonitrile, and also C₄₋₈ conjugated dienes, such as 1,3-butadiene (butadiene) and isoprene. In addition to or instead of the aforementioned monomers it is also possible for N-vinylformamide, N-vinylacetamide, VP, N-vinylimidazole, N-vinylcaprolactam, 2-vinylpyridine, 4-vinylpyridine or 2-methyl-5-vinylpyridine to be used for preparing the polymeric anionic dispersant. Also it is possible to hydrolyze formamide or acetamide groups possibly present in the polymeric anionic dispersant, to form primary amino groups. The aforementioned monomers generally form, in the anionic dispersants, the auxiliary monomers which, based on the total monomer amount, account for a fraction of less than 80%, frequently less than 50% and preferably less than 30% by weight. The polymeric anionic dispersants frequently contain none of the aforementioned monomers in copolymerized form.

It will be appreciated that it is also possible for the polymeric anionic dispersants to contain not only sulfonic and/or carboxylic acid groups but also groups protonated on the nitrogen and/or alkylated groups. In that case, however, it is essential for the dispersants to contain more sulfonic and/or carboxylic acid groups than groups protonated on the nitrogen and/or alkylated groups.

Suitable auxiliary monomers include the following monomers that are alkylated or protonated on the nitrogen: 1-vinylimidazole, 2-vinylimidazole, 2-vinylpyridine, 4-vinyl-pyridine, 2-methyl-5-methylpyridine, dialkylaminoalkyl acrylates, dialkylaminoalkyl methacrylates, dialkylaminoalkyl acrylamides, dialkylaminoalkyl methacrylamides, 3-aminopropyl vinyl ether, vinylamines or allylamines. Alkylation takes place by methods known to the skilled worker, such as by reaction with dimethyl sulfate, diethyl sulfate or methyl chloride. It will be appreciated that it is also possible to carry out the alkylation by means of aforementioned reagents at the polymer stage.

The K values of the polymeric anionic dispersants used are generally in a range from 10 to 350, frequently from 20 to 200 and often from 35 to 150.

By K values are meant, for the purposes of this text, generally the values measured by the method of Fikentscher, Cellulose-chemie, Vol. 13, pages 58 to 64 (1932) at 25° C. in 5% strength by weight aqueous sodium chloride solution. The concentration of the polymer under measurement (polymeric anionic dispersant or polyvinyllactam) in aforementioned sodium chloride solution is in each case 0.1 part by weight [g] per 100 parts by volume [ml] of 5% strength by weight aqueous sodium chloride solution.

It is important that the polymeric anionic dispersants are completely soluble in the aqueous reaction medium under reaction conditions in the quantity range actually employed, namely from 0.1% to 30% by weight, based on the total amount of said at least one N-vinyllactam I used for the polymerization, and are able to stabilize the droplets of N-vinyllactam that are present, and in particular the polyvinyllactam droplets formed, as a dispersedly separate phase.

It is further of importance that the polymeric anionic dispersants can be used optionally also in combination with so-called neutral protective colloids familiar to the skilled worker, such as polyvinyl alcohols, poly-N-vinyl-2-pyrrolidone, polyalkylene glycols, and also cellulose derivatives, starch derivatives or gelatin derivatives. The weight fraction of neutral protective colloids used optionally is, however, generally lower than the weight fraction of polymeric anionic dispersants and is often ≦5% by weight, ≦3% by weight or ≦1% by weight, based in each case on the total amount of said at least one N-vinyllactam I used for the polymerization.

In accordance with the invention the entirety of said at least one polymeric anionic dispersant, in combination if appropriate with the neutral protective colloids, can be introduced as an initial charge in the reaction medium. An alternative possibility is to include if appropriate only a portion of said at least one polymeric anionic dispersant, in combination if appropriate with the neutral protective colloids, in the initial charge in the reaction medium and to supply the remainder, if appropriate, or the entirety of said at least one polymeric anionic dispersant, in combination if appropriate with the neutral protective colloids, to the reaction medium under polymerization conditions.

As initiators for the free-radical polymerization it is possible to use water-soluble and water-insoluble peroxo compounds and/or azo compounds that are familiar to the skilled worker, such as, for example, alkali metal or ammonium peroxodisulfates, hydrogen peroxide, dibenzoyl peroxide, tert-butyl perpivalate, 2,2′-azobis(2,4-dimethyl-valeronitrile), tert-butyl peroxyneodecanoate, tert-butyl per-2-ethylhexanoate, di-tert-butyl peroxide, tert-butyl hydroperoxide, azobisisobutyronitrile, azobis(2-amidino-propane) dihydrochloride, 2,2′-azobis(2-methylpropionamidine) dihydrochloride (V-50 from Wako Chemicals GmbH, Neuss) or 2,2′-azobis(2-methylbutyronitrile). Also suitable are free-radical initiator mixtures or redox initiators, such as ascorbic acid/iron(II) sulfate/sodium peroxodisulfate, tert-butyl hydroperoxide/sodium disulfite or tert-butyl hydroperoxide/sodium hydroxymethanesulfonate, for example. The amount of said at least one free-radical initiator is from 0.01% to 0.25%, frequently from 0.05% to 0.2% and often from 0.1% to 0.2% by weight, based in each case on the total amount of said at least one N-vinyllactam I used for the polymerization.

Through the concomitant use of redox coinitiators, examples of which include benzoin, dimethylaniline and organically soluble complexes and salts of heavy metals, such as copper, cobalt, manganese, nickel and chromium or especially iron, it is possible to lower the half-lives of the stated peroxides, especially the hydroperoxides, so that, for example, tert-butyl hydroperoxide is active even at ≦70° C. in the presence of 5 ppm of copper(II) acetylacetonate.

Preference is given to using readily water-soluble azo initiators, such as 2,2′-azobis-(2-methylpropionamidine) dihydrochloride, for example.

The polymerization reaction is triggered by means of polymerization initiators which break down into free radicals. It is possible to employ all of the initiators known for polymerizing the N-vinyllactams I. Suitable examples include initiators which break down into free radicals and which at the temperatures chosen in each case possess half-lives of less than 3 hours. If the polymerization is conducted at different temperatures, by first initially polymerizing the N-vinyllactams I at a relatively low temperature and then completing polymerization at a significantly higher temperature, then it is advantageous to use at least two different initiators which possess a sufficient dissociation rate in the temperature range chosen in each case.

The polymerization is conducted at temperatures ≦70° C., often ≧20 and ≦70° C. or ≧45 and ≦65° C. and frequently ≧55 and ≧65° C. Frequently the polymerization reaction takes place under atmospheric pressure (1 bar absolute) or, if the polymerization is conducted in a closed system, under the autogenous pressure. In general the polymerization reaction takes place in the absence of oxygen, under a nitrogen atmosphere for example.

The polymerization can if appropriate also be conducted in the presence of molecular weight regulators, in order to tailor the molecular weight of the polymers. Examples of suitable polymerization regulators include 2-mercaptoethanols, mercaptopropanols, mercaptobutanols, thioglycolic acid, N-dodecyl mercaptan, tert-dodecyl mercaptan, thiophenol, mercaptopropionic acid, allyl alcohol and acetaldehyde. The molecular weight regulators are used in an amount, based on the vinyllactams I employed, of from 0% to 10%, or from 0% to 5%, or from 0% to 2%, by weight. In general, however, no molecular weight regulators are used in the process of the invention.

The process of the invention is frequently conducted in the presence of buffer substances, which are intended to suppress the hydrolysis of the N-vinyllactams I, especially at a pH <6. Examples of buffer substances used include sodium bicarbonate and sodium pyrophosphate, and also further compounds familiar to the skilled worker. Their amount, based on the aqueous reaction medium, is frequently from 0.01% to 2% by weight.

The pH of the aqueous reaction medium is generally in the range from 6 to 11, preference nevertheless being given to a pH ≦6.5 and ≧10 (measured in each case at 20 to 25° C.).

The aqueous reaction medium may additionally comprise further customary auxiliaries, such as biocides, viscosity regulators or defoamers, for example.

The process of the invention generally takes place such that the polymerization is carried out using ≧20% by weight, often >25% by weight and frequently ≧30% by weight of said at least one N-vinyllactam I, based on the total amount of the resulting polyvinyllactam dispersion.

It is essential that the entirety of said at least one N-vinyllactam I is polymerized in the process of the invention to a conversion of ≧90% by weight, often ≧95% by weight or frequently ≧98% by weight.

The process of the invention can take place either in accordance with the batch technique, with the entirety of said at least one N-vinyllactam I or introduced at the beginning, or by the feed technique.

If the polymerization takes place in batch mode, all of the components except for the free-radical initiator are introduced into the polymerization reactor at the start. Subsequently the aqueous polymerization mixture is heated to polymerization temperature, with stirring, and thereafter the free radical initiator is added, continuously or discontinuously.

In one preferred embodiment the process of the invention is carried out by means of a feed technique. In that case some or all of the reaction components are metered in whole or in part, in steps or continuously, together or in separate feed streams, into the aqueous reaction medium.

Advantageously at least a portion of said at least one organic or inorganic salt and of said at least one polymeric anionic dispersant and also if appropriate a portion of said at least one free-radical initiator and/or of said at least one N-vinyllactam I are introduced as an initial charge in the aqueous reaction medium, with stirring, and under polymerization conditions the remainders if appropriate of said at least one organic or inorganic salt and of said at least one polymeric anionic dispersant and also the entirety or remainder if appropriate of said at least one free-radical initiator and/or of said at least one N-vinyllactam I are metered in discontinuously or, in particular, continuously.

Following the polymerization operation the water-in-water dispersions obtained in the polymerization can be subjected to a physical or chemical aftertreatment.

For this purpose, for example, an additional 0.05 to 1.5% by weight, based on the total amount of said at least one N-vinyllactam I used for the polymerization, of at least one of the aforementioned free-radical initiators is metered continuously or discontinuously into the polymerization mixture under polymerization conditions in order to complete the polymerization. Advantageously, the entirety of the free-radical initiator is added discontinuously, in one lot, to the polymerization mixture under polymerization conditions in order to complete the polymerization.

Frequently the polymerization reaction proper is followed by aftertreatment of the resultant water-in-water dispersion by means of steam and/or nitrogen stripping for the purpose of removing highly volatile organic constituents. Methods of steam and/or nitrogen stripping are familiar to the skilled worker.

The water-in-water dispersions obtained are usually milky white and have a viscosity at 25° C. of from 5 to 90 000 mPas, often from 10 to 60 000 mPas and often from 15 to 30 000 mPas, measured in each case by the Brookfield method, spindle 4, 10 revolutions per minute.

The polyvinyllactams available through the process of the invention have K values ≧120, frequently ≧130 or even ≧140, measured by the method of Fikentscher (see above). The weight-average molecular weights of the polyvinyllactams available in accordance with the invention are situated within the range from 1 000 000 to 5 000 000 g/mol, frequently in the range from 1 500 000 to 4 000 000 g/mol and often in the range from 2 000 000 to 4 000 000 g/mol, determined in each case by means of standard methods of gel permeation chromatography.

The aqueous polyvinyllactam dispersions available in accordance with the invention can be placed directly on the market. An alternative possibility is for these dispersions to be freed from possibly disruptive accompanying components by means of oxidizing or reducing reagents, adsorption methods, such as the adsorption of impurities on selected media, such as on activated carbon, or by means of ultrafiltration methods. The aqueous polyvinyllactam dispersions available in accordance with the invention can alternatively again be converted into the corresponding polyvinyllactam powders by means of suitable drying methods, such as spray drying, freeze drying or roll drying, with the use of if appropriate of suitable auxiliaries, such as spray drying assistants or anticaking agents, for example.

It is significant that the high molecular mass polyvinyllactams available in accordance with the invention can be used with advantage, in the form of their water-in-water dispersions or in the form of their polymer powders, as a component in drug or cosmetic products, in adhesives, heat transfer fluids, in coating, thickener, adsorber, binder, laundry detergent, plastics, ceramics, refrigerant, ink or pigment formulations and also in metal quenching baths.

The process of the invention allows access to highly concentrated water-in-water dispersions of high molecular mass (K value ≧120) polyvinyllactams with good space/time yields. These dispersions are stable for many months, are of low viscosity despite the high polyvinyllactam content, and additionally have a negligibly small gel content, if any at all.

The examples which follow are intended to illustrate the invention, though without restricting it.

### EXAMPLES

Analysis

Determination of the Fikentscher K value was made at 25° C. by means of a 5% strength by weight solution of sodium chloride in deionized water, using an instrument from Schott, Mainz (capillary: Mikro-Ostwald; type: MO-Ic). The aqueous polyvinyllactam dispersion and a 5% strength by weight aqueous sodium chloride solution were mixed so that the resulting homogeneous solution had a polyvinyllactam content of 0.1 g per 100 ml of 5% strength by weight aqueous sodium chloride solution.

The polyvinyllactam content of the aqueous polyvinyllactam dispersion was determined by drying an aliquot thereof to constant weight in a drying oven at 140° C. The polyvinyllactam content is calculated from the corrected dry residue, based on the aliquot of aqueous polyvinyllactam dispersion used for drying. The corrected dry residue is the dry residue obtained after drying, minus the auxiliaries present alongside the polyvinyllactam in the aliquot of the aqueous polyvinyllactam dispersion used for drying, such as the amount of free-radical initiator and the amount of polymeric anionic dispersant, organic or inorganic salts, and other auxiliaries if appropriate.

The viscosity of the aqueous polyvinyllactam dispersion obtained was determined in accordance with ISO 2555 at 25° C. using a Brookfield instrument, model DV-II with spindle 4 at a rotary speed of 10 revolutions per minute.

### Example 1

A 1.5 l polymerization reactor with anchor stirrer was charged at 20 to 25° C. (room temperature) with
330 g of deionized water
63.4 g of sodium sulfate (anhydrous, Merck, Darmstadt)
148 g of a 20% strength by weight aqueous solution of a copolymer (of acrylic acid and vinylformamide in a 9:1 quantitative ratio, with subsequent hydrolysis and neutralization by means of aqueous sodium hydroxide solution, with a K value of 104 and a weight-average molecular weight of 1 070 000 g/mol).

Subsequently the pH of this reaction mixture was adjusted to 6.8 using a 5% strength by weight aqueous solution of sulfuric acid and thereafter this reaction mixture was heated with stirring (160 rpm) to 60° C. under a nitrogen atmosphere. After 60° C. had been reached, 10% by weight of the feed streams I and II, described below, were added to the reaction mixture, with stirring and retention of reaction temperature, and the system was stirred for 5 minutes under the abovementioned conditions.

Thereafter the remainders of feed streams I and II were metered in over the course of two hours, beginning simultaneously and with constant feed stream flows, into the reaction mixture, with stirring and retention of the reaction temperature. After the end of feed streams I and II polymerization was continued at 60° C. for three hours more.

Subsequently the reaction mixture was heated to 75° C. Thereafter feed stream III was added all at once to this polymerization mixture, which was left at this temperature with stirring for a further two hours. Subsequently the polymer dispersion was cooled to room temperature.

Feed stream I:
233.4 g of N-vinyl-2-pyrrolidone (from BASF AG, Ludwigshafen)

Feed stream 11 was an aqueous solution composed of:
0.35 g of 2,2′-azobis(2-methylpropionamidine) dihydrochloride (V-50, from Wako Chemicals GmbH, Neuss)
55.9 g of deionized water

Feed stream III was an aqueous solution composed of:
0.7 g of V-50
13 g of deionized water

The K value of the polyvinyllactam obtained was found to be 141, the viscosity of the resultant aqueous dispersion 10.3 Pas and the polyvinyllactam content of the aqueous dispersion 27.6% by weight.

### Example 2

Example 2 was prepared as for Example 1 but using the following raw materials and amounts thereof:

Initial charge:
464 g of deionized water
75 g of sodium sulfate
106 g of a 35.1% strength by weight aqueous solution of a copolymer (of maleic anhydride and methyl vinyl ether in a 1:1 quantitative ratio, with subsequent complete hydrolysis of the anhydride groups and neutralization by means of aqueous sodium hydroxide solution, with a K value of 90 and a weight-average molecular weight of 160 000 g/mol).

Feed stream I:
300 g of N-vinyl-2-pyrrolidone

Feed stream II was an aqueous solution composed of:
0.45 g of V-50
44.5 g of deionized water

Feed stream III was an aqueous solution composed of:
0.9 g of V-50
8 g of deionized water

The K value of the polyvinyllactam obtained was found to be 143, the viscosity of the resultant aqueous dispersion 27.5 Pas and the polyvinyllactam content of the aqueous dispersion 30.1% by weight.

### Example 3

Example 3 was prepared as for Example 1 but using the following raw materials and amounts thereof:

Initial charge:
383 g of deionized water
76.9 g of trisodium citrate dehydrate (Fluka, Germany)
89 g of a 37.8% strength by weight aqueous solution of a pure polyacrylic acid (neutralized with aqueous sodium hydroxide solution, having a K value of 80 and a weight-average molecular weight of 100 000 g/mol).

Feed stream I:
270 g of N-vinyl-2-pyrrolidone

Feed stream II was an aqueous solution composed of:
0.41 g of V-50
64.5 g of deionized water

Feed stream III was an aqueous solution composed of:
0.81 g of V-50
15 g of deionized water

The K value of the polyvinyllactam obtained was found to be 138, the viscosity of the resultant aqueous dispersion 7.5 Pas and the polyvinyllactam content of the aqueous dispersion 30.3% by weight.

### Example 4

Example 4 was prepared as for Example 1 but using the following raw materials and amounts thereof:

Initial charge:
565 g of deionized water
115.3 g of trisodium citrate dihydrate
143.8 g of a 35.1% strength by weight aqueous solution of a copolymer (of maleic anhydride and methyl vinyl ether in a 1:1 quantitative ratio, with subsequent complete hydrolysis of the anhydride groups and neutralization by means of aqueous sodium hydroxide solution, with a K value of 90 and a weight-average molecular weight of 160 000 g/mol).

Feed stream I:
405 g of N-vinyl-2-pyrrolidone

Feed stream II was an aqueous solution composed of:
0.61 g of V-50
96.8 g of deionized water

Feed stream III was an aqueous solution composed of:
1.22 g of V-50
22.5 g of deionized water

The K value of the polyvinyllactam obtained was found to be 144, the viscosity of the resultant aqueous dispersion 16.2 Pas and the polyvinyllactam content of the aqueous dispersion 31.9% by weight.

### Comparative Example

The comparative example was prepared as for Example 1 but without using sodium sulfate.

The experiment had to be abandoned after the addition of about 180 g of N-vinyl-2-pyrrolidone, owing to the resultant excessive viscosity.

1. A process for preparing a water-in-water dispersion of polyvinyllactam having a K value of ≦120 by free-radically initiated polymerization of at least one N-vinyllactam of general formula I 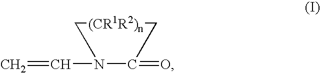
where
R¹, R² independently of one another are hydrogen and/or C₁-C₈ alkyl, and
n is an integer from 2 to 8,
in an aqueous reaction medium,
wherein said at least one N-vinyllactam I used for the polymerization is composed of at least 50% by weight of N-vinyl-2-pyrrolidone (R¹ and R² as hydrogen, n as 3), the polymerization temperature is ≦70° C. und the free-radically initiated polymerization of said at least one N-vinyllactam I takes place in the presence of
a) from 1% to 100% by weight, based on the saturation amount in the aqueous reaction medium, of at least one organic or inorganic salt,
b) from 0.1% to 30% by weight of at least one polymeric anionic dispersant, based on the total amount of said at least one N-vinyllactam I used for the polymerization, and
c) from 0.01% to 0.25% by weight of at least one free-radical initiator, based on the total amount of said at least one N-vinyllactam I used for the polymerization,
and the reaction conditions are chosen so that during the polymerization reaction at least a portion of said at least one N-vinyllactam I and of the polyvinyllactam formed therefrom by polymerization are present in the form of a separate phase in the aqueous reaction medium.

2. A process according to claim 1, wherein the polymerization is carried out using ≧20% by weight of said at least one N-vinyllactam I, based on the total amount of the resulting aqueous polyvinyllactam dispersion.

3. A process according to claim 1, wherein the polymerization takes place by the feed technique.

4. A process according to claim 3, wherein at least one portion of said at least one organic or inorganic salt and of said at least one polymeric anionic dispersant and if appropriate a portion of said at least one free-radical initiator and/or of said at least one N-vinyllactam I are introduced as an initial charge in the aqueous reaction medium and under polymerization conditions the remainders if appropriate of said at least one organic or inorganic salt and of said at least one polymeric anionic dispersant and also the entirety or remainder if appropriate of said at least one free-radical initiator and/or of said at least one N-vinyllactam I are metered in continuously.

5. A process according to claim 1, wherein the entirety of said at least one N-vinyllactam I is polymerized to a conversion of ≧90% by weight.

6. A process according to claim 5, wherein the polymerization is completed by metering additionally from 0.05% to 1.5% by weight, based on the total amount of said at least one N-vinyllactam I used for the polymerization, of at least one free-radical initiator into the polymerization mixture under polymerization conditions.

7. A process according to claim 1, wherein said at least one N-vinyllactam I used for the polymerization is exclusively N-vinyl-2-pyrrolidone.

8. A process according to claim 1, wherein as said at least one polymeric anionic dispersant homopolymers or copolymers of ethylenically unsaturated carboxylic or sulfonic acids and also their corresponding salts are used.

9. A process according to claim 1, wherein as said at least one salt the salt of an organic C₁ to C₁₅ carboxylic acid is used.

10. An aqueous polyvinyllactam dispersion obtainable by a process according to claim 1.

11. The method of using an aqueous polyvinyllactam dispersion according to claim 10 as a component in drug or cosmetic products, in adhesives, heat transfer fluids, in coating, thickener, adsorber, binder, laundry detergent, plastics, ceramics, refrigerant, ink or pigment formulations or in metal quenching baths.

12. A drug or cosmetic product, adhesive, heat transfer fluid, coating, thickener, adsorber, binder, laundry detergent, plastics, ceramics, refrigerant, ink or pigment formulation or metal quenching bath comprising at least one aqueous dispersion of polyvinyllactam having a K value ≧120 and a polyvinyllactam content of ≧20% by weight, based on the total amount of the aqueous polyvinyllactam dispersion.
